(19) United States
(12) Patent Application Publication
(10) Pub. No.: US 2014/0277307 A1
(43) Pub. Date: Sep. 18, 2014

(54) Companion animal convective air blankets

(71) Applicant: ADROIT MEDICAL SYSTEMS, INC, LOUDON, TN (US)

(72) Inventors: Scott Gammons, Loudon, TN (US); Clifford E. Gammons, Loudon, TN (US); Jack Payne, Knoxville, TN (US); Jeff Stickley, Dartmouth, MA (US)

(73) Assignee: ADROIT MEDICAL SYSTEMS, INC, LOUDON, TN (US)

(21) Appl. No.: 14/214,911

(22) Filed: Mar. 15, 2014

Related U.S. Application Data
(60) Provisional application No. 61/787,329, filed on Mar. 15, 2013.
(60) Provisional application No. 61/876,453, filed on Sep. 11, 2013.

(51) Int. Cl. A61F 7/00

(52) U.S. Cl. CPC A61F 7/0097
USPC 607/107

(57) ABSTRACT

Apparatus for convective air therapy blankets for companion animals. Convective air blankets include two sheets joined together to form an inflatable chamber. One of the two sheets is air permeable to exhaust conditioned air toward the animal. The blankets includes fenestrations, slits, straps, and inlet ports positioned to accommodate the anatomy and surgical needs of companion animals. Blankets with fenestrations have an I-shaped opening to allow passage of a limb. The opposite end of the blanket has a slit to drape the blanket around the neck and head of the animal. Blankets with a central access port provide surgical access of the animal's underbody. Blankets also have integral straps for securing the blanket to the animal. Inlet ports are provided to allow connection to an air supply from either side of the blanket or to an edge where the two sheets come together.

### CROSS-REFERENCE TO RELATED APPLICATIONS

This application claims the benefit of U.S. Provisional Application No. 61/787,329, filed Mar. 15, 2013, and U.S. Provisional Application No. 61/787,329, filed Sep. 11, 2013.

### STATEMENT REGARDING FEDERALLY SPONSORED RESEARCH OR DEVELOPMENT

Not Applicable

### BACKGROUND

1. Field of Invention

This invention pertains to convective air therapy blankets. More particularly, this invention pertains to a thermal therapy device that provides convective warming to a companion animal during the perioperative period.

2. Description of the Related Art

Companion animal surgery is becoming more common as demand for companion animal care increases. Unlike human surgery, companion animal surgery presents unique issues and problems.

Most companion animals have fur that insulates their body from changing environmental conditions. The animal's coat is crucial for thermoregulation of the animal's body temperature. However, most surgical procedures require removing the animal's fur from at least a portion of their body. Without the insulation provided by their coat, the animal becomes susceptible to hypothermia, particularly during surgery conducted in cooled surgical theaters. Surgical drapes and blankets are often used to keep the animal warm perioperatively, but the blankets are often cumbersome and interfere with the surgical procedures.

Convective air blankets are commonly used to provide warming of human patients. These blankets are used with humans placed on a flat surface, such as a hospital bed or operating table. Also, the blankets are used on humans when they are in the prone or supine position, with their limbs typically supported on the flat surface. In contrast, companion animals are often operated on with a contoured or V-shaped surgical platform. It is not uncommon for animals to have one or more limbs elevated above the surgical table.

### BRIEF SUMMARY

According to one embodiment of the present invention, convective air blankets for companion animals are provided. The blankets are configured for use with companion animals during the perioperative period of surgical procedures. The blankets are also configured for therapeutic use. The blankets have a first sheet attached to a second sheet with at least one air inlet for introducing conditioned air. The first sheet is air impermeable, such as a non-woven fabric with a plastic film or coating. The second sheet is air permeable, such as a non-woven fabric with a desired porosity or an impermeable sheet with apertures, holes, or beaks that allow air to pass.

In one embodiment, the blanket includes a fenestration that allows a limb of the animal to protrude through the blanket. The fenestration provides surgical and orthopedic access to the animal's extremities. In one such embodiment, the fenestration has an I-shape. A long line of weakness is bounded by a pair of perpendicular lines of weakness. The lines of weakness, in one embodiment, are perforations in the first and second sheets that allow the sheets to be separated easily. The first and second sheets are sealed together around the fenestration such that the air chamber defined by the first and second sheets is sealed at the fenestration.

In another embodiment, the blanket includes an access port that allows surgical access to the animal's torso. Such blankets are suited for soft-tissue procedures performed on the body of the companion animal. In yet another embodiment, the blanket is a reversible warming device configured to be placed either under the animal or over the animal. In one such embodiment, an inlet port is positioned on each sheet to allow attachment of the air supply hose when the blanket is either under or over the animal. In still another embodiment, the blanket is a warming basket that partially encloses the animal. In another embodiment, the blanket includes a convective air warming tube.

The convective air blankets include a side inlet port. The side inlet port feature is suitable for use with all types of blankets, including those for companion animals and human patients. The side inlet port feature is particularly suited for smaller sized blankets in which the air supply hose can deform the inflated shape of the blanket depending upon how the hose is supported and draped.

The air supply hose connects to the inlet port with the hose connector in the plane of the blanket. In this way the end of the hose will not twist the blanket when the weight of the hose causes the outlet connector to assume a horizontal position. The inlet port is folded with half of the port on one outer layer and the other half on the other outer layer of the blanket. A hole or notch is formed in the blanket to allow the port to be connected to both surfaces of the blanket. In use, the folded inlet port is unfolded sufficiently to allow the hose outlet connector to be received by the inlet port. When the blanket inflates, the ballooning effect of the blanket supports the two halves of the inlet port such that the hose connector maintains engagement with the inlet port.

In another embodiment the side inlet port is a sleeve or funnel defined by an opening in a corner of the blanket. The two sheets are joined by the peripheral seam extending along the sides of the sleeve such that a tubular sleeve is formed. A strap is attached to the sleeve such that the strap is wrapped around the sleeve after the sleeve receives a nozzle. The ends of the strap are connected together to secure the nozzle in the sleeve and provide a substantially air-tight seal around the nozzle. In one embodiment a barrier sheet is disposed between the first and second sheets. The barrier sheet extends from the distal end of the nozzle along the channels extending away from the channel. The barrier sheet is configured so that the nozzle is positioned in the sleeve between the barrier sheet and the air impermeable sheet. In this way air from the nozzle is directed into the inflatable chamber before it is exhausted through the air permeable sheet, thereby aiding in the distribution of the conditioned air throughout the chamber and through the air permeable sheet.

Another feature of the convective air blankets is that they are fluid repellant. The fluid repellant feature prolongs the usable life of the blanket by maintaining the blanket's functionality when the blanket is exposed to blood and other bodily fluids during surgery. The fluid repellant feature is suitable for use with all types of blankets, including those for companion animals and human patients.

The blankets include non-woven material with a coating disposed on the outside surface. The coating provides the fluid repellant feature. In order to thermally weld the layers of material to form the blanket, a third layer of a thermoplastic film is disposed between the two outer layers of material. The layers are welded with a particular pattern to form an air bladder between the two outer sheets with the inner film fusing to the two outer sheets.

### BRIEF DESCRIPTION OF THE SEVERAL VIEWS OF THE DRAWINGS

The above-mentioned features will become more clearly understood from the following detailed description read together with the drawings in which:

FIG. 1 is a perspective view of one embodiment of a convective air blanket used with an animal on an operating table.

FIG. 2 is a top plan view of one embodiment of an Henle-extremity convective air blanket for a large sized animal.

FIG. 3 is an exploded diagram showing the convective air blanket of FIG. 2.

FIG. 4 is a top plan view of another embodiment of an Henle-extremity convective air blanket for a medium sized animal.

FIG. 5 is a top plan view of another embodiment of an Henle-extremity convective air blanket for a small sized animal.

FIG. 6 is a top plan view of one embodiment of a soft-tissue convective air blanket for a large sized animal.

FIG. 7 is a top plan view of another embodiment of a soft-tissue convective air blanket for a medium sized animal.

FIG. 8 is a top plan view of another embodiment of a soft-tissue convective air blanket for a small sized animal.

FIG. 9 is a top plan view of another embodiment of a soft-tissue convective air blanket for an extra small sized animal.

FIG. 10 is a top plan view of one embodiment of an over-under convective air blanket for a large sized animal.

FIG. 11 is a top plan view of one embodiment of an over-under convective air blanket for a medium sized animal.

FIG. 12 is a top plan view of one embodiment of an over-under convective air blanket for a small sized animal.

FIG. 13 is a partial exploded view of one corner of the over-under convective air blanket of FIG. 10 showing the inlet ports.

FIG. 14 is a plan view of one embodiment of a convective air warming basket/blanket.

FIG. 15 is a view of the inflated warming basket blanket of FIG. 14 shown in the deployed, inflated configuration.

FIG. 16 is a plan view of one embodiment of a convective air warming tube blanket.

FIG. 17 is a plan view of another embodiment of a soft-tissue convective air blanket with a side inlet port for a medium sized animal.

FIG. 18 is a plan view of another embodiment of a soft-tissue convective air blanket with a side inlet port for an extra small sized animal.

FIG. 19 is an exploded diagram showing the convective air blanket of FIG. 18.

FIG. 20 is a plan view of another embodiment of a convective air warming basket/blanket with a side inlet port.

FIG. 21 is a plan view of another embodiment of a convective air warming tube blanket with a side inlet port.

FIG. 22 is a plan view of another embodiment of a convective air warming blanket with another embodiment of a side inlet port.

FIG. 23 is an exploded diagram showing the convective air blanket of FIG. 22.

### DETAILED DESCRIPTION

Apparatus for convective warming of a companion animal during the perioperative period is disclosed. The convective warming device is a blanket that is generally indicated as 100, with particular embodiments and variations shown in the figures and described below having an alphabetic suffix, for example, 100-A, 100-B. The convective warming device 100 is intended to be connected to at least one air supply that provides conditioned air to the device 100.

FIG. 1 illustrates a perspective view of a convective air blanket 100 used with an animal 102 on an operating table 112. The table 112 is covered with a drape 106 with the animal 102 on the drape 106 and covered with the convective air device 100. The leg 114 of the animal 102 extends through a fenestration 110 in the convective air device 100. In this way the animal 102 is kept warm by the convective air device 100 while allowing the animal's limb 114 to be exposed for a surgical procedure.

The convective air device 100 has a sheet 108 that is permeable to air. An air supply hose 104 is connected to the device 100. The hose 104 provides conditioned air. The convective air device 100 receives the conditioned air and directs it toward the animal 102 through the sheet 108.

FIG. 2 illustrates a top plan view of one embodiment of an Henle-extremity convective air blanket 100-A for a large sized animal 102. FIG. 3 illustrates an exploded diagram showing the convective air blanket 100-A of FIG. 2.

The convective air blanket 100-A includes a first sheet 108-A and a second sheet 202-A. The first sheet 108-A is an air permeable fabric, such as a non-woven fabric with a desired permeability or breathability. In another embodiment, the first sheet 108-A is a material with a multitude of openings spaced across the surface of the sheet 108-A. Such openings, in various embodiments, being holes or beaks. The first sheet 108-A is positioned proximate the animal 102 so that conditioned air exhausted through the sheet 108-A is directed toward the animal 102.

The second sheet 202-A is an air impermeable fabric, such as a non-woven fabric with a polymer film or coating to inhibit air flow through the fabric. The second sheet 202-A is positioned away from the animal 102.

The two sheets 108-A, 202-A are joined together with a peripheral seam 204-A that is air tight. The two sheets 108-A, 202-A and the peripheral seam 204-A define an air bladder or chamber that receives conditioned air through one of the inlet ports 302 in the sheet 202-A. The inlet port 302 is a line of weakness, such as a perforation formed with an X pattern. The inlet ports 302 are surrounded with a connector 212-A. The connector is a stiff board, such as cardboard, with a central opening. When a nozzle of an air supply hose 104 is to be mated with a connector 212-A, the inlet port 302 is opened by breaking the lines of weakness, thereby allowing air to enter the blanket 100-A.

With multiple air inlets 302 in a blanket 100, there is sometimes a need to seal one of the air inlets 302 after the inlet 302 is opened. When the blanket 100 is first in use, one of the air inlets 302 has the lines of weakness broken so that air can be introduced into the blanket 100. Sometimes it is desirable to move the hose 104 from the first air inlet 302 to a second air inlet 302. In such a case, the first air inlet 302 must be resealed to avoid loss of air through the opened inlet 302. In one embodiment, an inlet seal 308 has an adhesive side that is placed over the connector 212-A at the open, but undesired, air inlet 302. In this way the unused air inlet 302 is sealed.

Within the boundary of the peripheral seam 204-A are a series of channel seams 206-A that define channels 226 between the lines of the channel seams 206-A and between the channel seams 206-A and the peripheral seam 204-A. The channels 226 direct the conditioned air from the inlets 302 through the convective air device 100-A to ensure even distribution of conditioned air throughout the device 100-A. The spacing of the channel seams 206-A relative to each other and the peripheral seam 204-A is such that the channels 226 are narrow. Narrow channels 226 result in a blanket 100 that is thin and has a low profile. Considering the rounded body of companion animals 102, the low profile allows medical personnel better access to the animal at the site of interest.

The blanket 100-A has a head end 222 and a tail end 224. The head end 222 includes a slit 210 that is, in one embodiment, defined by a line of weakness and, in another embodiment, is a slit that separates the sheets 108-A, 202-A proximate the head of the animal 102. The peripheral seam 204-A has a notch 208 that bounds the slit 210. In this way, when the slit 210 is opened to accommodate the head of the animal 102 or to allow passage of tubes and instruments, the chamber integrity is maintained by the notch 208.

Near the tail end 224 is a fenestration 110-A. The fenestration 110 is an opening through which a limb 114 passes. In the illustrated embodiment, the fenestration 110-A has a central seam 218 that is bounded by two perpendicular side seams 214. The group of seams 218, 214 form an I-shape. The central seam 218 and the side seams 214 are wide seams that overlay lines of weakness 304, 306 in the second sheet 202-A. In this way the fenestration 110-A maintains the air tightness of the chamber, even when the lines of weakness 304, 306 are broken to allow access through the blanket 100-A. The fenestration 110-A is bounded by a pair of thin seams 216 parallel to the central seam 218. The thin seams 216 prevent the flaps defined by the I-shaped fenestration 110-A from being inflated by air and ballooning.

In the illustrated embodiment, the fenestration 100-A has a central seam 218 that is greater than one-half the width of the blanket 100-A. A fenestration 110-A of such size allows great latitude in its use. Only the portion of the lines of weakness 304, 306 located where the limb 114 is desired to protrude from the blanket 100-A need be broken. The remainder of the lines of weakness 304, 306 do not need to be broken, thereby aiding maintaining a warm air zone proximate the animal 102.

FIG. 3 illustrates a fenestration card 310 that frames the fenestration 110. The card 310 is also suitable for framing the access port 602 in the soft-tissue convective air blankets 100-D, 100-E, 100-F, 100-G illustrated in FIGS. 6-9. The card 310 is a stiff member with an opening 312 sized to accommodate the fenestration 110 or access port 602. The card 310 is attached to the sheet 202-A with an adhesive 314. In one embodiment, the adhesive 314 is a double-sided tape that is initially affixed to one side of the card 310. When the card 310 is desired to be used, the protective cover on the adhesive 314 is removed and the card 310 is then stuck to the sheet 202-A. The illustrated embodiment shows an adhesive 316 on the outer side of the card 310. The outer adhesive 316 is a double-sided tape that is initially affixed to the outer side of the card 310 for attaching a separate drape used to cover the animal 102 and/or blanket 100. In the illustrated embodiment, the adhesives 314, 316 are shown as short strips. In another embodiment, the adhesive 314, 316 surrounds the opening in the card 310.

In another embodiment, the opening 312 is smaller than the fenestration 110 or access port 602. In this way the bottom surface of the card 310 overlaps the edge of the fenestration 110 or access window 602 and the adhesive 314 is positioned to adhere to the animal 102 and/or the sheet 202.

FIG. 4 illustrates a top plan view of another embodiment of an Henle-extremity convective air blanket 100-B for a medium sized animal 102. The blanket 100-B is similar to that of the blanket 100-A shown in FIG. 2 except that the distance between the head end 222 and the tail end 224 is shorter to accommodate animals 102 that are medium sized. The fenestration 110-A is substantially the same for the medium blanket 100-B.

FIG. 5 illustrates a top plan view of another embodiment of an Henle-extremity convective air blanket 100-C for a small sized animal 102. Like the blanket 100-B shown in FIG. 4, the blanket 100-C shown in FIG. 5 is even smaller to accommodate animals 102 that are small sized.

The fenestration 110-B in the small blanket 100-C has a shorter central seam 218′ and side seams 214′. Because the fenestration 110-B is smaller, the sealing seams 216 shown in FIG. 2 are not necessary for the smaller blanket 100-C.

FIG. 6 illustrates a top plan view of one embodiment of a soft-tissue convective air blanket 100-D for a large sized animal 102. FIG. 7 illustrates a top plan view of another embodiment of a soft-tissue convective air blanket 100-E for a medium sized animal 102. FIG. 8 illustrates a top plan view of another embodiment of a soft-tissue convective air blanket 100-F for a small sized animal 102. FIG. 9 illustrates a top plan view of another embodiment of a soft-tissue convective air blanket 100-G for a extra small sized animal 102. The blankets 100-D, 100-E, 100-F, 100-G each include a split 210 at the head end 222 to accommodate the head of the animal 102 or to allow passage of tubes and instruments.

The soft-tissue convective air blankets 100-D, 100-E, 100-F, 100-G each include a peripheral seam 204-D to -G that is near the edge of the sheet 202-D to -G. Each peripheral seam 204-D to -G bounds an access port or window 602. The access port 602 is inside an access port seam 604 that joins the two sheets 108-D to -G, 202-D to -G with an air-tight seal. The access port 602 is defined by a line of weakness, such as a perforation through the two sheets 108-D to -G, 202-D. Breaking all or part of the perforation defining the access port 602 allows access to the midsection of the animal 102 under the blanket 100-D to -G.

The soft-tissue convective air blankets 100-D, 100-E, 100-F, 100-G each include a peripheral seam 204-D to -G that bounds channel seams 206-D to -G that are configured to accommodate the size of animal 102 used with the blanket 100-D to -G. The channels 226 defined by the channel seams 206-D to -G are narrow to minimize the thickness of the blankets 100-D to -G around the access port 602. Access to the animal 102 is enhanced by the separation between the edge of the access port 602 and the access port seam 604, which covers a portion of the animal 102 adjacent the access port 602 with two, uninflated sheets 108, 202. The thin covering allows medical personnel good access to the animal 102 at the site of interest inside the access port 602 because of the minimal thickness of the two sheets 108, 202.

FIG. 10 illustrates a top plan view of one embodiment of an over-under convective air blanket 100-H for a large sized animal 102. FIG. 11 illustrates a top plan view of one embodiment of an over-under convective air blanket 100-I for a medium sized animal 102. FIG. 12 illustrates a top plan view of one embodiment of an over-under convective air blanket 100-J for a small sized animal 102.

The over-under convective air blankets 100-H, 100-I, 100-J each include a peripheral seam 204-H to -J that bounds channel seams 206-H to -J that are configured to accommodate the size of animal 102 used with the blanket 100-H to -J. The channel seams 206-H to -J are spaced closely together such that the channels 226 remain inflated with the blanket 100-H to -J positioned under the animal 102 and conditioned air supplied to the blanket 100-H to -J.

The blanket 100-I illustrated in FIG. 11 also includes straps or ties 1102. A perforated slit 1104 is cut near the slit 210 for the neck. The perforated slit 1104 is broken and the ties 1102, which remain connected near the neck slit 210, are located to tie the blanket 100-I around the animal to secure the blanket 100-I to the animal. In other embodiments the blankets 100-H, 100-J illustrated in FIGS. 10 and 12 also have ties 1102.

FIG. 13 illustrates a partial exploded view of one corner of the over-under convective air blanket 100-H showing the inlet ports 302-A, 302-B and their corresponding connectors 212-A1, 201-A2. Because the blankets 100-H to -J are configured to fit under or over the animal 102, in one embodiment an inlet port 302-A, 302-B is positioned on each sheet 202-H to -I, 108-H to -I, respectively. In this way an inlet port 302 is accessible regardless of the position of the blanket 100-H to -I being under or over the animal 102.

With the two inlet ports 302-A, 302-B coincident, the perforations for the lines of weakness are made with a single operation during manufacturing, thereby aiding in the manufacturability of the blankets 100-H to -J. With respect to the small blanket 100-J, the inlet ports are either located in opposite corners on one sheet 202-J, as illustrated, on opposite corners with one port 302 on each sheet 108-J, 202-J, or in the same corner on each sheet 108-J, 202-J as shown in FIG. 11.

FIG. 14 illustrates a plan view of one embodiment of a convective air warming basket blanket 100-K. FIG. 15 illustrates a perspective view of the inflated warming basket blanket 100-K of FIG. 14 shown in the deployed, inflated configuration.

The channel seams 206-K1 are arranged to form a channel 226 that has a donut or ring shape. The inside of the donut defined by the channel seams 206-K1 has a group of seams 206-K2 that form a tufted cushion inside the donut shape. An animal 102 supported on the basket 100-K rests on the center of the donut and cradled by the donut.

Two embodiments for wrapping the basket 100-K around the animal 102 are illustrated in FIG. 14. One embodiment has three straps 1402 that extend from each side of the basket 100-K with the opposite ends being of sufficient length to be tied together to draw the basket 100-K together. In one such embodiment, the three straps 1402 are continuous and separate from the sheet 108-K, 202-K. In another such embodiment, the straps 1402 are attached to one or both of the sheets 108-K, 202-K.

Another embodiment includes a cord 1406 that is threaded through openings or eyelets 1408 in the sheets 108-K, 202-K. The free ends of the cord 1406 pass through a toggle 1404 that allows the cord 1406 to be cinched to draw together the edges of the blanket 100-K to form a basket shape as shown in FIG. 15.

FIG. 16 illustrates a plan view of one embodiment of a convective air warming tube blanket 100-L. The blanket 100-L includes a pair of sheets 108-L, 202 that are substantially the same size. The peripheral seam 204-L is in the shape of an outline of the letter U. Inside the peripheral seam 204-L are two dart seams 206-L2 positioned at the outer corners of the U-shape. Between the legs of the U-shape is another seam 206-L1 that keeps the two sheets 108-L, 202 together when the blanket 100-L is inflated. When the tube blanket 100-L receives conditioned air through the connector 212-A the blanket 100-L inflates into a U-shaped tube. The animal 102 is cradled in the area 1602 between the legs of the U-shaped tube.

In one embodiment, the tube blanket 100-L is partially wrapped around the animal 102 by a pair of straps 1402 that extend from the sides of the blanket 100-L. In one such embodiment, the three straps 1402 are continuous and separate from the sheet 108-L, 202. In another such embodiment, the straps 1402 are attached to one or both of the sheets 108-L, 202.

FIG. 17 illustrates a plan view of another embodiment of a soft-tissue convective air blanket 100-E1 with a side inlet port 212-B for a medium sized animal. The embodiment of the blanket 100-E1 shown in FIG. 17 is similar to the blanket 100-E shown in FIG. 7 with the exception of the configuration of the inlet port 212-B. The inlet port 212-B is folded in half with one half on the top side adjacent the top sheet 202-E1 and the other half of the inlet port 212-B on the bottom side adjacent the bottom sheet 108-E1. The sheets 202-E1, 108-E1 have an opening 1702 through which the inlet port 212-B passes. The peripheral seam 204-E has a straight portion 1704 in the corner over which the inlet port 212-B is positioned. Adjacent the opening 1702 and outside the inflated boundary of the peripheral seam 204-E is a joining seam 1706. The joining seam 1706 keeps the sheets 202-E1, 108-E1 together to aid in the engagement of the outlet connector of the air supply hose to the inlet port 212-B.

FIG. 18 illustrates a plan view of another embodiment of a soft-tissue convective air blanket 100-G1 with a side inlet port 212-B for a extra small sized animal. The embodiment of the blanket 100-G1 shown in FIG. 18 is similar to the blanket 100-G shown in FIG. 9 with the exception of the configuration of the inlet port 212-B. The inlet port 212-B is folded in half with one half on the top side adjacent the top sheet 202-G1 and the other half of the inlet port 212-B on the bottom side adjacent the bottom sheet 108-G1. The sheets 202-G1, 108-G1 have an opening 1702 through which the inlet port 212-B passes.

FIG. 19 illustrates an exploded diagram showing the convective air blanket 100-G1 of FIG. 18. The side inlet port 212-B is shown partially folded above the three openings 1702-A, 1702-B, 1702-C in the sheets 202-G1, 108-G1, 1902. The three openings 1702-A, 1702-B, 1702-C are aligned when the are joined to form the blanket 100-G1. In this way, on half of the inlet port 212-B is attached to the sheet 202-G1 and the other half of the port 202-A is attached to the sheet 108-G1.

The inlet port 212-B is configured to allow the outlet connector of the air supply hose to remain in the same plane as the blanket 100-G1, thereby avoiding any twisting or deformation of the blanket 100-G1 when the air supply hose is connected to a top mounted inlet port 212-A and the air supply hose is supported by the same surface upon which the blanket 100-G1 rests. When the blanket 100-G1 is in the deflated state, the inlet port 212-B is folded and the blanket 100-G1 is a planar structure that can be folded into a compact arrangement for storage and deployment. When the blanket 100-G1 is deployed, the inlet port 212-B is unfolded sufficiently to allow the outlet connector of the air supply hose to engage the inlet port 212-B. When the blanket 100-G1 is in the inflated state, the separation of the sheets 202-G1, 108-G1 by the air and the outlet connector in the inlet port 212-B serve to keep the inlet port 212-B in a mostly unfolded configuration.

The side inlet port 212-B is suitable for use with other types of convective air blankets 100, such as shown in the other figures and for blankets configured for human use. In particular, the side inlet port 212-B avoids twisting or deformation of the smaller sized blankets, such as the blanket 100-G1 illustrated.

FIG. 19 also illustrates one embodiment of achieving the fluid repellant feature of the blankets 100. One or both of the outer sheets 202-G1, 108-G1 are non-woven material with a coating 1904 on one side. The coating 1904 prevents the material of the sheets 202-G1, 108-G1 from absorbing fluid in contact with the outer surface of the sheets 202-G1, 108-G1. The opposite sides of the sheets 202-G1, 108-G1 have no coating 1904. The intermediate sheet 1902 is a polymer film that fuses the sheets 202-G1, 108-G1 together to form the various seals 204-G, 206-G. In one embodiment, the intermediate sheet 1902 is a thermosetting film that fuses the sheets 202-G1, 108-G1 together when heat is applied to the blanket 100-G1 to form a laminated structure. In one such embodiment, the intermediate sheet 1902 is the same size or slightly smaller than the smallest of the two sheets 202-G1, 108-G1 and the heat is applied with a die having a heated shape corresponding to the seams 204-G, 206-G. In another such embodiment, the intermediate sheet 1902 is sized and shaped to conform to the pattern of the various seals 204-G, 206-G.

In another embodiment of the fluid repellant feature, one or both of the outer sheets 202-G1, 108-G1 are material, such as paper or a non-woven fabric, with a coating 1904 that sufficiently saturates the material such that the two sheets 202-G1, 108-G1 can be thermally welded together without the need for the intermediate sheet 1902. In another embodiment, one or both of the outer sheets 202-G1, 108-G1 are non-woven material with a first coating on one side suitable for thermal welding the two sheets 202-G1, 108-G1 together without the need for the intermediate sheet 1902. The other side of the sheets 202-G1, 108-G1 have a second coating 1904 that is fluid repellant, such as a coating that is hydrophobic.

The fluid repellant feature is suitable for all types of convective air blankets 100 that are subject to fluids during use. The fluid repellant feature allows the blanket 100 to be used for extended periods and/or to be re-used.

In one embodiment, an apparatus for providing convective air includes a first sheet 202-G1 that is air impermeable; a second sheet 108-G1 that is air permeable, said first and second sheets 202-G1, 108-G1 joined at a peripheral seam 204-G to form an air chamber; and an air inlet 212 into said air chamber, said air inlet 212 configured to receive a supply of conditioned air that is exhausted from said second sheet 108-G1, said air inlet 212-B defined by a sheet of stiff material having a fold, said fold defining a first and second portion of said air inlet 212-B, said first portion of said air inlet 212-B attached to said first sheet 202-G1 and said second portion of said air inlet attached to said second sheet 108-G1. In one such embodiment, the two sheets each include an opening 1702, and said fold of said air inlet 212-C is disposed in said openings 1702. In another such embodiment, said air inlet 212-C is positioned proximate an edge of said first and second sheets 202-G1, 108-G1, and said fold of said air inlet 212-C is disposed in a notch 2002 formed in said first and second sheets 202-G1, 108-G1.

In another embodiment, an apparatus for providing convective air includes a first sheet 202-G1 that is air impermeable, said first sheet 202-G1 having a coating 1904 on one surface, said coating 1904 being water resistant; a second sheet 108-G1 that is air permeable, said second sheet having a coating 1904 on one surface, said coating 1904 being water resistant; a third sheet 1902 that is a polymer for fusing together said first and second sheets 202-G1, 108-G1 in selected regions, said third sheet 1902 disposed between said first and second sheets, said coating on each of said first and second sheets located on an opposite side of each of said first and second sheets 202-G1, 108-G1 from a side proximate said third sheet 1902, said first and second sheets joined at a peripheral seam 204-G to form an air chamber; and an air inlet 212 into said air chamber, said air inlet 212 configured to receive a supply of conditioned air that is exhausted from said second sheet 108-G1.

FIG. 20 illustrates a plan view of another embodiment of a convective air warming basket/blanket 100-K1 with a side inlet port 212-B. The embodiment of the blanket 100-K1 shown in FIG. 20 is similar to the blanket 100-K shown in FIG. 14 with the exception of the configuration of the inlet port 212-B. FIG. 21 illustrates a plan view of another embodiment of a convective air warming tube blanket 100-L1 with a side inlet port 212-B. The embodiment of the blanket 100-L1 shown in FIG. 21 is similar to the blanket 100-L shown in FIG. 16 with the exception of the configuration of the inlet port 212-B.

In the embodiments shown in FIGS. 20 and 21, the inlet port 212-B is positioned adjacent the edge of the blanket 100-K1, 100-L1. The edge of the blanket 100-K1, 100-L1 adjacent the inlet port 212-B has a notch 2002 in the sheets 202-K1, 108-K1, 202-L1, 108-L1. The notch 2002 is an area with no material of the sheets 202-K1, 108-K1, 202-L1, 108-L1 outside the edge of the peripheral seam 204-K, 204-L. The fold of the inlet port 212-B is immediately adjacent to where the peripheral seam 204-K, 204-L would pass under the inlet port 212-B. In this way the outlet connector of the air supply hose directly engages the inlet port 212-B without interference with the sheets 202-K1, 108-K1, 202-L1, 108-L1.

In another embodiment, the outer edge of the blanket 100 adjacent the side inlet port 212-B is folded over the peripheral seam 204 and the folded side inlet port 212-B is positioned over the folded material of the blanket 100. In this way the notch 2002 is formed by the folded material between the peripheral seam 204 and the edge of the sheets 202, 108.

FIG. 22 illustrates a plan view of another embodiment of a convective air warming blanket 100-M with another embodiment of a side inlet port 212-C. FIG. 23 illustrates an exploded diagram of the convective air blanket 100-M with the location of the various seams 204-M, 206-M, 2206 shown on the sheets 108-M, 202-M. The illustrated blanket 100-M is an over-under blanket such as illustrated in FIGS. 10-12. The illustrated embodiment of the side inlet port 212-C is a funnel or sleeve-type inlet that receives a wide range of air supply nozzles and is suitable for use on a wide range of convective air blankets 100, including those discussed herein.

The blanket 100-M includes a first sheet 108-M and a second sheet 202-M. The first sheet 108-M is air permeable and is configured to be in contact with the animal 102 such that conditioned air is directed toward the animal 102. In one embodiment the first sheet 108-A is a non-woven fabric with the desired permeability or breathability. In another embodiment the first sheet 108-A is a material with a multitude of openings spaced across the surface of the sheet 108-A. Such openings, in various embodiments, being holes or beaks.

The second sheet 202-M is air impermeable and is the outer sheet that is positioned away from the animal 102. In one embodiment the second sheet 202-M is a thin material, such as a nonwoven fabric, that has a polymer film or coating to inhibit air flow through the material.

The two sheets 108-M, 202-M are almost the same size with the outer sheet 202-M being slightly narrower. A peripheral seam 204-M defines an inflatable chamber between the two sheets 108-M, 202-M. Within the boundary of the peripheral seam 204-M are a series of channel seams 206-M that define channels 226 between the lines of the channel seams 206-M and between the channel seams 206-M and the peripheral seam 204-M. The channels 226-M direct the conditioned air from the inlet port 212-C through the convective air device 100-M to ensure even distribution of conditioned air throughout the device 100-M and through the sheet 108-M.

One corner 2204 of the blanket 100-M is configured to receive a nozzle from an air supply hose 104 from a convective air supply. A funnel or sleeve 2212 is formed from the two sheets 108-M, 202-M that are joined at the sides by the peripheral seam 204-M. That is, the air chamber defined by the peripheral seam 204-M is open at the end of the sleeve 2212. The sides of the sheets 202-M, 108-M defining the sleeve 2212 are parallel, as are the portions of the peripheral seam 204-M in the seam, or the sides are at an angle, which further aids the insertion of an air hose nozzle into the funnel or sleeve 2212.

A strap 2202 is attached to the sleeve 2212 and the strap 2202 is cinched after the nozzle is inserted between the two sheets 108-M, 202-M in order to seal the sleeve 2212 around the nozzle, thereby providing an air-tight connection to the nozzle regardless of size. The strap 2202 is attached to the sleeve 2212 with a connector 2304, such as a snap, rivet, or sewn connection. In one embodiment the strap 2202 is a rolled fabric that is sewn. In other embodiments the strap 2202 is a string, cord, or similar structure that is suitable for tying or use with a connector, clamp, or toggle that joins the ends of the strap 2202 to hold the sleeve 2212 around the nozzle.

In the illustrated embodiment a barrier sheet 2208 is disposed between the two sheets 108-M, 202-M next to the inlet port 212-C. A piece of tape 2302 is adhered to the barrier sheet 2208 between the top sheet 202-M and the barrier sheet 2208. A distal end of the tape 2302 is folded over and adhered to the outside of the bottom sheet 108-M. In this way when the nozzle from the conditioned air supply is inserted into the sleeve 2212, the nozzle will be positioned between the barrier sheet 2208 and the top sheet 202-M. In this way the air flow from the nozzle will be directed between the barrier sheet 2208 and the top sheet 202-M. The conditioned air is directed to the full chamber defined by the peripheral seal 204-M before the conditioned air can escape from the air permeable sheet 108-M. An inlet seam 2206 is positioned in the chamber in the direct air path from the sleeve 2212. In this way the conditioned air is further distributed within the inflatable air chamber to ensure even exhaustion of air through the sheet 108-M.

In one embodiment various seams 206-M have an associated stress relief seam 2210. The stress relief seams 2210 are positioned at an end of the channel seams 206-M that have an extended space that would allow the chamber to expand and pull the end of the seam 206-M apart. The stress relief seam 2210 is a semicircular seam that joins the sheets 108-M, 202-M such that the stress from an inflated chamber is spread out across the arcuate length of the stress relief seam 2210 instead of having the stress concentrated at an end point of a channel seam 206-M. In this way the blanket 100-M is suited for multiple inflations over a period of time without leaking.

The blankets 100 include various functions. The function of allowing access to a limb 114 of an animal 102 while the animal 102 is covered with the warming blanket 100-A, 100-B, 100-C is implemented, in various embodiments, by the fenestration 110 as illustrated in FIGS. 1-5.

The function of allowing access to the body of the animal 102 while the animal 102 is covered with the warming blanket 100-D, 100-E, 100-F, 100-G is implemented, in various embodiments, by the access port 602 as illustrated in FIGS. 6-9.

The function of cradling an animal 102 in a warming blanket 100-K, 100-L is implemented, in various embodiments, by the blankets illustrated in FIGS. 14-16. In one such embodiment, a basket 100-K cradles the animal 102. In another embodiment, a tube blanket 100-L cradles the animal 102 in the area 1602 between the legs of the U-shaped tube.

The function of connecting an air hose 104 in a parallel plane as the blanket 100 is implemented, in various embodiments, by the side inlet ports 212-B, 212-C, such as illustrated in FIGS. 17-23. The side inlet ports 212-B, 212-C allow the devices 100 to maintain their shape when connected to a conditioned air supply because the weight of the air hose 104 does not pull the inlet port 212 away from its normal deployed position.

The function of accepting various sized air hoses 104 is implemented, in one embodiment, by the side inlet port 212-C that includes the funnel or sleeve 2212, such as depicted in FIGS. 22, 23.

From the foregoing description, it will be recognized by those skilled in the art that a companion animal convective air blanket 100 has been provided. The blanket 100 has several different embodiments that provide solutions to different problems encountered when using convective air devices 100 with animals 102. In one embodiment the blanket 100-A, 100-B, 100-C includes a fenestration 110 configured to allow a limb 114 of animal 102 to extend through the blanket 100-A, 100-B, 100-C. In another embodiment the blanket 100-D, 100-E, 100-E, 100-G allows access to the soft tissue portion of the animal 102 through an access port 602. In a third embodiment the blanket 100-H, 100-I, 100-J is an under-over blanket that has an inlet port 212-A1, 212-A2 on each side of the blanket 100-H, 100-I, 100-J. In a fourth embodiment the blanket 100-K, 100-L has a basket-shape that cradles or nests the animal 102. In a fifth embodiment the blanket 100-E1, 100-G1, 100-K1, 100-L1 includes a side inlet port 212-B that attaches at the peripheral seam 204. In a sixth embodiment the blanket 100-M includes a side inlet port 212-C that has a funnel or sleeve 2212 that receives various sized nozzles from an air hose 104. In a seventh embodiment the blanket 100-G1 includes the feature of fluid repellant sheets 202-G1, 108-G1 where the outside of one or both sheets 202, 108 include a coating 1904 that is fluid repellant.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. An apparatus for providing convective air to a companion animal, said apparatus comprising:
a first sheet that is impermeable to conditioned air,
a second sheet that is permeable to conditioned air, said first and second sheets joined at a peripheral seam to form an air chamber;
an air inlet into said air chamber, said air inlet configured to receive a supply of conditioned air that is exhausted through said second sheet; and
a fenestration defined by three lines of weakness in said first and second sheets, two of said three lines of weakness being parallel and spaced apart, a third one of said three lines of weakness perpendicular to and bisecting said two of said three lines of weakness, whereby said three lines of weakness form an I-shape, said fenestration surrounded by a fenestration seal that isolates said air chamber from said three lines of weakness.

2. The apparatus of claim 1 wherein said fenestration further includes a first seam joining a pair of distal ends of each one of said two of said three lines of weakness on one side of said third one of said three lines of weakness, and a second seam joining another pair of distal ends of each one of said two of said three lines of weakness on an opposite side of said third one of said three lines of weakness.

3. The apparatus of claim 1 further including a first slit extending from an edge of said first sheet and a second slit extending from an edge of said second sheet, said first slit coincident with said second slit, said first and second slits outside of said peripheral seam, said first and second slits defining a slot configured to receive a body part of the companion animal.

4. The apparatus of claim 1 further including a pair of straps, each one of said pair of straps defined by a first slit parallel to an edge of said first sheet and a second slit parallel to an edge of said second sheet, each one of said first slits coincident with a corresponding one of said second slits, a portion of said first sheet between each one of said first slits and said edge of said first sheet bonded to a corresponding portion of said second sheet between each one of said second slits and said edge of said second sheet.

5. The apparatus of claim 1 wherein said air inlet is defined by a sheet of stiff material having a fold, said fold defining a first and second portion of said air inlet, said first portion of said air inlet attached to said first sheet and said second portion of said air inlet attached to said second sheet.

6. The apparatus of claim 1 wherein said air inlet is defined by a sleeve extending from said air chamber, said sleeve defined by said first and second sheets wherein a portion of said peripheral seam joins said first and second sheets, said sleeve configured to receive a nozzle supplying conditioned air, and said sleeve including a pair of opposing straps configured to wrap around said sleeve when said nozzle is received in said sleeve, thereby securing said nozzle in said sleeve.

7. The apparatus of claim 6 further including a barrier sheet disposed between said first and second sheets, said barrier sheet extending from a distal end of said sleeve into said air chamber, said barrier sheet attached to said second sheet proximate said second sheet such that said sleeve receives said nozzle between said barrier sheet and said first sheet.

8. The apparatus of claim 1 wherein said second sheet has an outer surface with a coating, and said coating being water resistant.

9. An apparatus for providing convective air to a companion animal, said apparatus comprising:
a first sheet that is impermeable to conditioned air,
a second sheet that is permeable to conditioned air, said first and second sheets joined at a peripheral seam to form an air chamber,
an air inlet into said air chamber, said air inlet configured to receive a supply of conditioned air that is exhausted from said second sheet; and
an access port defined by a line of weakness in said first and second sheets, said access port positioned within the boundary of said peripheral seam, said first and second sheets joined together adjacent said line of weakness with an access port seal that is positioned between said line of weakness and said peripheral seam wherein said access port is isolated from said air chamber.

10. The apparatus of claim 9 further including a first slit extending from an edge of said first sheet and a second slit extending from an edge of said second sheet, said first slit coincident with said second slit, said first and second slits outside of said peripheral seam, said first and second slits defining a slot configured to receive a body part of the companion animal.

11. The apparatus of claim 9 further including a pair of straps, each one of said pair of straps defined by a first slit parallel to an edge of said first sheet and a second slit parallel to an edge of said second sheet, each one of said first slits coincident with a corresponding one of said second slits, a portion of said first sheet between each one of said first slits and said edge of said first sheet bonded to a corresponding portion of said second sheet between each one of said second slits and said edge of said second sheet.

12. The apparatus of claim 9 wherein said air inlet is defined by a sheet of stiff material having a fold, said fold defining a first and second portion of said air inlet, said first portion of said air inlet attached to said first sheet and said second portion of said air inlet attached to said second sheet.

13. An apparatus for providing convective air to a companion animal, said apparatus comprising:
a first sheet that is impermeable to conditioned air,
a second sheet that is permeable to conditioned air, said first and second sheets joined at a peripheral seam to form an air chamber,
an air inlet into said air chamber, said air inlet configured to receive a supply of conditioned air that is exhausted from said second sheet; and
a means for cradling the companion animal between a opposing edges of said second sheet.

14. The apparatus of claim 13 wherein said means for cradling includes a cord threaded proximate each one of said opposing edges of said second sheet, said cord having a pair of distal ends extending from said second sheet whereby pulling said pair of distal ends away from said second sheet draws said opposing edges together to form a basket shape.

15. The apparatus of claim 14 further including a connector positioned proximate said pair of distal ends of said cord; said connector securing said pair of distal ends of said cord together whereby said cord cinches said second sheet.

16. The apparatus of claim 13 wherein said means for cradling includes a pair of straps extending from opposite edges of said second sheet, said pair of ties having a length sufficient to fasten said pair of straps together.

17. An apparatus for providing convective air to a patient, said apparatus comprising:
a first sheet that is air impermeable;
a second sheet that is air permeable, said first and second sheets joined at a peripheral seam to form an air chamber; and
an air inlet configured to receive a nozzle connected to a supply of conditioned air that is exhausted from said second sheet, said air inlet configured to extend from said apparatus without obstruction when said apparatus is positioned over said patient and when said apparatus is positioned under said patient.

18. The apparatus of claim 17 wherein said air inlet includes a first air inlet in said first sheet and a second air inlet in said second sheet, said second air inlet positioned coaxial with said first air inlet, said first inlet having a first inlet opening positioned over a first line of weakness in said first sheet, said second inlet having a second inlet opening positioned over a second line of weakness in said first sheet, wherein one of said first and second lines of weakness is broken when an associated one of said first and second air inlets is selected to receive said nozzle.

19. The apparatus of claim 17 wherein said air inlet is defined by a sheet of stiff material having a fold, said fold defining a first and second portion of said air inlet, wherein said first portion of said air inlet is attached to said first sheet and said second portion of said air inlet is attached to said second sheet.

20. The apparatus of claim 17 wherein said air inlet is defined by a sleeve extending from said air chamber, said sleeve defined by said first and second sheets wherein a portion of said peripheral seam joins said first and second sheets on opposing sides of said sleeve, and said sleeve configured to receive the nozzle supplying conditioned air.
